(19) United States
(12) Patent Application Publication
(10) Pub. No.: US 2018/0243253 A1
(43) Pub. Date: Aug. 30, 2018

(54) FORMULATIONS CONTAINING OMEGA-3 FATTY ACIDS OR ESTERS THEREOF AND MAQUI BERRY EXTRACT AND THERAPEUTIC USES THEREOF

(71) Applicant: FOCUS LABORATORIES, INC., North Little Rock, AR (US)

(72) Inventors: Patrick H. WITHAM, Eugene, OR (US); Edward Lee PAUL, JR., Wrightsville Beach, NC (US)

(21) Appl. No.: 15/936,322

(22) Filed: Mar. 26, 2018

Related U.S. Application Data
(63) Continuation of application No. PCT/US2018/020006, filed on Feb. 27, 2018.
(60) Provisional application No. 62/463,932, filed on Feb. 27, 2017.

(51) Int. Cl. A61K 31/202; A61K 36/185; A61K 9/48; A61P 27/14; A61P 27/12; A61P 27/06; A61P 27/04

(52) U.S. Cl. CPC A61K 31/202; A61K 36/185; A61K 9/48; A61K 2300/00; A61P 27/12; A61P 27/06; A61P 27/04; A61P 27/14

(57) ABSTRACT

Provided herein are methods, formulations, and compositions related to omega-3 fatty acids or esters thereof and maqui berry extract. Methods, formulations, and compositions as described herein provide for treatment of ocular diseases or disorder

### CROSS-REFERENCE

This application is a continuation of PCT International Application No. PCT/US2018/020006, filed Feb. 27, 2018, which claims the benefit of U.S. Provisional Application No. 62/463,932 filed on Feb. 27, 2017, each of which are incorporated herein by reference in their entirety.

### BACKGROUND

Millions of individuals suffer from an ocular disease or disorder costing billions of dollars a year in healthcare. For example, dry eye disease alone affects 20 million individuals. Further, ocular diseases or disorders are prevalent with aging populations and are associated with other diseases, for example, diabetes and degenerative diseases.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Provided herein are compositions comprising: (a) omega-3 fatty acids or esters thereof; and (b) maqui berry extract, wherein a ratio of the omega-3 fatty acids or esters thereof to the maqui berry extract is about 12:1 to about 150:1. Further provided herein are compositions, wherein the ratio of the omega-3 fatty acids or esters thereof to the maqui berry extract is about 30:1 to about 120:1. Further provided herein are compositions, wherein the ratio of the omega-3 fatty acids or esters thereof to the maqui berry extract is about 40:1 to about 80:1. Further provided herein are compositions, wherein the composition is formulated for oral administration. Further provided herein are compositions, wherein the composition is formulated as a capsule, cachet, or tablet. Further provided herein are compositions, wherein the capsule is a soft gel capsule. Further provided herein are compositions, wherein the omega-3 fatty acids or esters thereof comprise eicosapentaenoic acid (EPA). Further provided herein are compositions, wherein the omega-3 fatty acids or esters thereof comprise docosahexaenoic acid (DHA). Further provided herein are compositions, wherein the omega-3 fatty acids or esters thereof comprise eicosapentaenoic acid (EPA) and docosahexaenoic acid (DHA). Further provided herein are compositions, wherein a ratio of the EPA to the DHA is in a range of 0.7:1 to 5:1. Further provided herein are compositions, wherein a ratio of the EPA to the DHA is in a range of 1:1 to 4:1. Further provided herein are compositions, wherein a ratio of the EPA to the DHA is in a range of 2:1 to 3:1. Further provided herein are compositions, wherein a ratio of the EPA to the DHA is 2:1. Further provided herein are compositions, wherein a ratio of the EPA to the DHA is 1.5:1.

Provided herein are methods of treating an ocular disease or disorder in a subject comprising: administering to the subject omega-3 fatty acids or esters thereof and maqui berry extract, wherein a ratio of the omega-3 fatty acids or esters thereof to the maqui berry extract is about 12:1 to about 150:1. Further provided herein are methods of treating an ocular disease or disorder in a subject, wherein the ocular disease or disorder is selected from a group consisting of allergies, glaucoma, cataract, corneal disease, vitreo-retinal diseases, optic nerve diseases or disorders, oculosystemic diseases and disorders, uvea diseases or disorders, and diabetic eye disease. Further provided herein are methods of treating an ocular disease or disorder in a subject, wherein the ocular disease or disorder is dry eye. Further provided herein are methods of treating an ocular disease or disorder in a subject, wherein a dose of the omega-3 fatty acids or esters thereof is administered to the subject one time to four times a day. Further provided herein are methods of treating an ocular disease or disorder in a subject, wherein a dose of the omega-3 fatty acids or esters thereof is administered to the subject four times a day. Further provided herein are methods of treating an ocular disease or disorder in a subject, wherein a dose of the maqui berry extract is administered to the subject one time to four times a day. Further provided herein are methods of treating an ocular disease or disorder in a subject, wherein a dose of the maqui berry extract is administered to the subject four times a day. Further provided herein are methods of treating an ocular disease or disorder in a subject, wherein the ratio of the omega-3 fatty acids or esters thereof to the maqui berry extract is about 30:1 to about 120:1. Further provided herein are methods of treating an ocular disease or disorder in a subject, wherein the ratio of the omega-3 fatty acids or esters thereof to the maqui berry extract is about 40:1 to about 80:1. Further provided herein are methods of treating an ocular disease or disorder in a subject, wherein the omega-3 fatty acids or esters thereof comprise eicosapentaenoic acid (EPA). Further provided herein are methods of treating an ocular disease or disorder in a subject, wherein the omega-3 fatty acids or esters thereof comprise docosahexaenoic acid (DHA). Further provided herein are methods of treating an ocular disease or disorder in a subject, wherein the omega-3 fatty acids or esters thereof comprise eicosapentaenoic acid (EPA) and docosahexaenoic acid (DHA). Further provided herein are methods of treating an ocular disease or disorder in a subject, wherein a ratio of the EPA to the DHA is in a range of 0.7:1 to 5:1. Further provided herein are methods of treating an ocular disease or disorder in a subject, wherein a ratio of the EPA to the DHA is in a range of 1:1 to 4:1. Further provided herein are methods of treating an ocular disease or disorder in a subject, wherein a ratio of the EPA to the DHA is in a range of 2:1 to 3:1. Further provided herein are methods of treating an ocular disease or disorder in a subject, wherein a ratio of the EPA to the DHA is 2:1. Further provided herein are methods of treating an ocular disease or disorder in a subject, wherein a ratio of the EPA to the DHA is 1.5:1. Further provided herein are methods of treating an ocular disease or disorder in a subject, wherein the omega-3 fatty acids or esters thereof and the maqui berry extract are formulated for oral administration. Further provided herein are methods of treating an ocular disease or disorder in a subject, wherein the omega-3 fatty acids or esters thereof and the maqui berry extract are formulated as a capsule, cachet, or tablet. Further provided herein are methods of treating an ocular disease or disorder in a subject, wherein the capsule is a soft gel capsule.

### BRIEF DESCRIPTION OF THE DRAWINGS

FIG. 1 illustrates a soft gel capsule comprising omega-3 fatty acids or esters thereof and maqui berry extract.

### DETAILED DESCRIPTION

Ocular disease or disorders are common human illnesses affecting millions of individuals. Treating ocular diseases or disorders, in some instances, is challenging due to the eye's blood ocular barriers and efflux pumps. Further, in some instances, treatments for ocular diseases or disorders only treat the symptoms and not the underlying cause of the ocular disease or disorder.

Provided herein are methods, formulations, and compositions for treating ocular diseases or disorders. Methods, formulations, and compositions for treating ocular diseases or disorders as described herein comprise omega-3 fatty acids or esters thereof and maqui berry extract. Further provided herein are methods, formulations, and compositions for treating ocular diseases or disorders comprising omega-3 fatty acids or esters thereof and maqui berry extract for treating inflammation underlying the ocular diseases or disorder and associated symptoms.

### Omega-3 Fatty Acids

Provided herein, in some embodiments, are methods, formulations, and compositions related to omega-3 fatty acids or esters thereof and maqui berry extract, wherein a daily dosage of omega-3 fatty acids or esters thereof comprises about 500 mg to about 4000 mg. In some embodiments, a daily dosage of omega-3 fatty acids or esters thereof comprises at least or about 500 mg, 750 mg, 1000 mg, 1200 mg, 1400 mg, 1600 mg, 1800 mg, 2000 mg, 2200 mg, 2400 mg, 2600 mg, 2800 mg, 3000 mg, 3200 mg, 3400 mg, 3600 mg, 3800 mg, 4000 mg, or more than 4000 mg. In some embodiments, a daily dosage of omega-3 fatty acids or esters thereof comprises about 400 mg to about 800 mg, about 400 mg to about 1,200 mg, about 400 mg to about 1,600 mg, about 400 mg to about 2,000 mg, about 400 mg to about 2,400 mg, about 400 mg to about 2,800 mg, about 400 mg to about 3,200 mg, about 400 mg to about 3,600 mg, about 400 mg to about 4,000 mg, about 400 mg to about 4,400 mg, about 400 mg to about 4,800 mg, about 800 mg to about 1,200 mg, about 800 mg to about 1,600 mg, about 800 mg to about 2,000 mg, about 800 mg to about 2,400 mg, about 800 mg to about 2,800 mg, about 800 mg to about 3,200 mg, about 800 mg to about 3,600 mg, about 800 mg to about 4,000 mg, about 800 mg to about 4,400 mg, about 800 mg to about 4,800 mg, about 1,200 mg to about 1,600 mg, about 1,200 mg to about 2,000 mg, about 1,200 mg to about 2,400 mg, about 1,200 mg to about 2,800 mg, about 1,200 mg to about 3,200 mg, about 1,200 mg to about 3,600 mg, about 1,200 mg to about 4,000 mg, about 1,200 mg to about 4,400 mg, about 1,200 mg to about 4,800 mg, about 1,600 mg to about 2,000 mg, about 1,600 mg to about 2,400 mg, about 1,600 mg to about 2,800 mg, about 1,600 mg to about 3,200 mg, about 1,600 mg to about 3,600 mg, about 1,600 mg to about 4,000 mg, about 1,600 mg to about 4,400 mg, about 1,600 mg to about 4,800 mg, about 2,000 mg to about 2,400 mg, about 2,000 mg to about 2,800 mg, about 2,000 mg to about 3,200 mg, about 2,000 mg to about 3,600 mg, about 2,000 mg to about 4,000 mg, about 2,000 mg to about 4,400 mg, about 2,000 mg to about 4,800 mg, about 2,400 mg to about 2,800 mg, about 2,400 mg to about 3,200 mg, about 2,400 mg to about 3,600 mg, about 2,400 mg to about 4,000 mg, about 2,400 mg to about 4,400 mg, about 2,400 mg to about 4,800 mg, about 2,800 mg to about 3,200 mg, about 2,800 mg to about 3,600 mg, about 2,800 mg to about 4,000 mg, about 2,800 mg to about 4,400 mg, about 2,800 mg to about 4,800 mg, about 3,200 mg to about 3,600 mg, about 3,200 mg to about 4,000 mg, about 3,200 mg to about 4,400 mg, about 3,200 mg to about 4,800 mg, about 3,600 mg to about 4,000 mg, about 3,600 mg to about 4,400 mg, about 3,600 mg to about 4,800 mg, about 4,000 mg to about 4,400 mg, about 4,000 mg to about 4,800 mg, or about 4,400 mg to about 4,800 mg. In some embodiments, a daily dosage of omega-3 fatty acids or esters thereof is in a range of about 1000 mg to about 3000 mg. In some embodiments, a daily dosage of omega-3 fatty acids or esters thereof comprises about 2400 mg. In some embodiments, a daily dosage of omega-3 fatty acids or esters thereof comprises about 2250 mg.

The daily dosage of omega-3 fatty acids or esters thereof, in some embodiments, is provided as one or more doses of omega-3 fatty acids or esters thereof. In some embodiments, at least or about 1, 2, 3, 4, 5, 6, 7, 8, 9, 10, or more than 10 doses of omega-3 fatty acids or esters thereof is provided. In some embodiments, doses of omega-3 fatty acids or esters thereof are provided in a range of about 1 to about 10, about 2 to about 9, about 3 to about 8, or about 4 to about 7 doses of omega-3 fatty acids or esters thereof. In some embodiments, about 4 doses of omega-3 fatty acids or esters thereof are provided.

The dose of omega-3 fatty acids or esters thereof, in some embodiments, is in a range of about 250 mg to about 1000 mg. In some embodiments, the dose of omega-3 fatty acids or esters thereof comprises at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of omega-3 fatty acids or esters thereof. In some embodiments, the dose of omega-3 fatty acids or esters thereof comprises about 100 mg to about 200 mg, about 100 mg to about 300 mg, about 100 mg to about 400 mg, about 100 mg to about 500 mg, about 100 mg to about 600 mg, about 100 mg to about 700 mg, about 100 mg to about 800 mg, about 100 mg to about 900 mg, about 100 mg to about 1,000 mg, about 100 mg to about 1,200 mg, about 200 mg to about 300 mg, about 200 mg to about 400 mg, about 200 mg to about 500 mg, about 200 mg to about 600 mg, about 200 mg to about 700 mg, about 200 mg to about 800 mg, about 200 mg to about 900 mg, about 200 mg to about 1,000 mg, about 200 mg to about 1,200 mg, about 300 mg to about 400 mg, about 300 mg to about 500 mg, about 300 mg to about 600 mg, about 300 mg to about 700 mg, about 300 mg to about 800 mg, about 300 mg to about 900 mg, about 300 mg to about 1,000 mg, about 300 mg to about 1,200 mg, about 400 mg to about 500 mg, about 400 mg to about 600 mg, about 400 mg to about 700 mg, about 400 mg to about 800 mg, about 400 mg to about 900 mg, about 400 mg to about 1,000 mg, about 400 mg to about 1,200 mg, about 500 mg to about 600 mg, about 500 mg to about 700 mg, about 500 mg to about 800 mg, about 500 mg to about 900 mg, about 500 mg to about 1,000 mg, about 500 mg to about 1,200 mg, about 600 mg to about 700 mg, about 600 mg to about 800 mg, about 600 mg to about 900 mg, about 600 mg to about 1,000 mg, about 600 mg to about 1,200 mg, about 700 mg to about 800 mg, about 700 mg to about 900 mg, about 700 mg to about 1,000 mg, about 700 mg to about 1,200 mg, about 800 mg to about 900 mg, about 800 mg to about 1,000 mg, about 800 mg to about 1,200 mg, about 900 mg to about 1,000 mg, about 900 mg to about 1,200 mg, or about 1,000 mg to about 1,200 mg. In some embodiments, the dose of omega-3 fatty acids or esters thereof comprises about 560 mg. In some embodiments, the dose of omega-3 fatty acids or esters thereof comprises at least or about 400 mg/g, 450 mg/g, 500 mg/g, 550 mg/g, 600 mg/g, 650 mg/g, 700 mg/g, 750 mg/g, 800 mg/g, 850 mg/g, 900 mg/g, 1000 mg/g or more than 1000 mg/g.

In some embodiments, omega-3 fatty acids or esters thereof comprise eicosapentaenoic acid (EPA), docosahexaenoic acid (DHA), or a combination thereof. In some embodiments, the omega-3 fatty acids or esters thereof comprise EPA. In some embodiments, the omega-3 fatty acids or esters thereof comprise DHA. In some embodiments, a ratio of the EPA to the DHA is in a range of about 0.7:1 to 5:1. In some embodiments, the ratio of the EPA to the DHA is at least or about 0.2:1, 0.25:1, 0.5:1, 0.7:1 0.75:1, 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1, 5:1, 5.25:1, 5.5:1, 5.75:1, or 6:1. In some embodiments, the ratio of the EPA to the DHA is about 0.2:1 to about 6:1, about 0.25:1 to about 5.75:1, about 5:1 to about 5.5:1, about 0.7:1 to about 5:1, about 0.75:1 to about 4.75:1, about 1:1 to about 4:1, or about 2:1 to about 3:1. In some embodiments, the ratio of the EPA to the DHA is about 2:1 to about 3:1. In some embodiments, the ratio of the EPA to the DHA is 2:1. In some embodiments, the ratio of the EPA to the DHA is 1.5:1.

In some embodiments, the omega-3 fatty acids or esters thereof comprise at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of EPA. In some embodiments, the omega-3 fatty acids or esters thereof comprise a range of about 10 mg to about 3000 mg, about 20 mg to about 2800 mg, about 40 mg to about 2400 mg, about 60 mg to about 2000 mg, about 80 mg to about 1600 mg, about 100 mg to about 1000, about 120 mg to about 800 mg, or about 200 mg to about 600 mg of EPA. In some embodiments, the omega-3 fatty acids or esters thereof comprise at least or about 300 mg/g, 350 mg/g, 400 mg/g, 450 mg/g, 500 mg/g, 550 mg/g, 600 mg/g, 650 mg/g, 700 mg/g or more of EPA. In some embodiments, the omega-3 fatty acids or esters thereof comprise at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of DHA. In some embodiments, the omega-3 fatty acids or esters thereof comprise a range of about 10 mg to about 3000 mg, about 20 mg to about 2800 mg, about 40 mg to about 2400 mg, about 60 mg to about 2000 mg, about 80 mg to about 1600 mg, about 100 mg to about 1000, about 120 mg to about 800 mg, or about 200 mg to about 600 mg of DHA. In some embodiments, the omega-3 fatty acids or esters thereof comprise at least or about 100 mg/g, 150 mg/g, 200 mg/g, 250 mg/g, 300 mg/g, 350 mg/g, 400 mg/g, 450 mg/g or more of DHA.

In some embodiments, the DHA or the EPA is derived from sources including, but not limited to, fish, algae, squid, yeast, and vegetable sources.

In some embodiments, the omega-3 fatty acids or esters thereof comprise additional omega-3 fatty acids or esters thereof in addition to EPA and DHA. For example, the omega-3 fatty acids or esters thereof comprise alpha-linolenic acid. In some embodiments, the additional omega-3 fatty acids or esters thereof comprise at least or about 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, or more than 100 mg.

Provided herein are methods, formulations, and compositions related to omega-3 fatty acids or esters thereof and maqui berry extract, wherein the omega-3 fatty acids or esters thereof comprise omega-3 fatty acids or esters thereof in various forms. For example, the omega-3 fatty acids or esters thereof are in a triglyceride form, re-esterified triglyceride concentrates, an ethyl ester form, a free fatty acid form, or a phospholipids form. In some embodiments, the omega-3 fatty acids or esters thereof are in triglyceride form.

In some embodiments, the triglycerides comprise at least or about 20%, 25%, 30%, 35%, 40%, 45%, 50%, 55%, 60%, 65%, 70%, 75%, 80%, 85%, 90%, or more than 90% of the formulation or composition comprising omega-3 fatty acids or esters thereof and maqui berry extract. In some embodiments, the triglycerides are in a range of about 5% to about 100%, about 10% to about 90%, 20% to about 80%, or about 30% to about 70% of the formulation or composition comprising omega-3 fatty acids or esters thereof and maqui berry extract.

### Maqui Berry

Described herein are methods, formulations, and compositions comprising omega-3 fatty acids or esters thereof and maqui berry extract. In some embodiments, the maqui berry extract comprises maqui berry of the plant genus Aristotelia. In some embodiments, the maqui berry extract comprises maqui berry of the plant Aristotelia chilensis.

In some embodiments, anthocyanins of the maqui berry are used. Exemplary anthocyanins of the maqui berry include, but are not limited to, delphinidin 3-sambubioside-5-glucoside; delphinidin 3,5-diglucoside; cyanidin 3-sambubioside-5-glucoside; cyanidin 3,5-diglucoside; delphinidin 3-sambubioside; delphinidin 3-glucoside; cyanidin 3-sambubioside; and cyanidin 3-glucoside. In some embodiments, the anthocyanins are isolated from the maqui berry. In some embodiments, the anthocyanins are isolated from dried leaves, whole berry, or seed of the maqui berry. In some embodiments, the anthocyanins are isolated from one or more parts of the maqui berry by physically removing a piece of the maqui berry fruit, such as by grinding the leaf of the fruit. In some embodiments, the anthocyanins are isolated from the berry by using extraction procedures well known in the art including, but not limited to, use of organic solvents such as C₁-C₈ alcohols, C₁-C₈ alkyl polyols, C₁-C₈ alkyl ketones, C₁-C₈ alkyl ethers, acetic acid C₁-C₈ alkyl esters, and chloroform, inorganic solvents, inorganic acids, and inorganic bases.

Provided herein, in some embodiments, are methods, formulations, and compositions related to omega-3 fatty acids or esters thereof and maqui berry extract, wherein a daily dosage of maqui berry extract comprises about 10 mg to about 300 mg. In some embodiments, the daily dosage of the maqui berry extract comprises at least or about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg or more than 300 mg. In some embodiments, the daily dosage of the maqui berry extract comprises about 10 mg to about 25 mg, about 10 mg to about 50 mg, about 10 mg to about 75 mg, about 10 mg to about 100 mg, about 10 mg to about 150 mg, about 10 mg to about 200 mg, about 25 mg to about 50 mg, about 25 mg to about 75 mg, about 25 mg to about 100 mg, about 25 mg to about 150 mg, about 25 mg to about 200 mg, about 50 mg to about 75 mg, about 50 mg to about 100 mg, about 50 mg to about 150 mg, about 50 mg to about 200 mg, about 75 mg to about 100 mg, about 75 mg to about 150 mg, about 75 mg to about 200 mg, about 100 mg to about 150 mg, about 100 mg to about 200 mg, or about 150 mg to about 200 mg. In some embodiments, the daily dosage of the maqui berry extract is in a range of about 20 mg to about 80 mg. In some embodiments, the daily dosage of the maqui berry extract comprises about 60 mg.

The daily dosage of maqui berry extract, in some embodiments, is provided as one or more doses of the maqui berry extract. In some embodiments, at least or about 1, 2, 3, 4, 5, 6, 7, 8, 9, 10, or more than 10 doses of the maqui berry extract is provided. In some embodiments, doses of the maqui berry extract are provided in a range of about 1 to about 10, about 2 to about 9, about 3 to about 8, or about 4 to about 7 doses of omega-3 fatty acids or esters thereof. In some embodiments, about 4 doses of the maqui berry extract are provided.

The dose of the maqui berry extract, in some embodiments, is in a range of about 5 mg to about 100 mg. In some embodiments, the dose of the maqui berry extract comprises at least or about 5 mg, 10 mg, 15 mg, 20 mg, 25 mg, 30 mg, 35 mg, 40 mg, 45 mg, 50 mg, 55 mg, 60 mg, 65 mg, 70 mg, 80 mg, 85 mg, 90 mg, 95 mg, 100 mg, or more than 100 mg of the maqui berry extract. In some embodiments, the dose of the maqui berry extract comprises about 5 mg to about 10 mg, about 5 mg to about 15 mg, about 5 mg to about 20 mg, about 5 mg to about 25 mg, about 5 mg to about 30 mg, about 5 mg to about 40 mg, about 5 mg to about 50 mg, about 10 mg to about 15 mg, about 10 mg to about 20 mg, about 10 mg to about 25 mg, about 10 mg to about 30 mg, about 10 mg to about 40 mg, about 10 mg to about 50 mg, about 15 mg to about 20 mg, about 15 mg to about 25 mg, about 15 mg to about 30 mg, about 15 mg to about 40 mg, about 15 mg to about 50 mg, about 20 mg to about 25 mg, about 20 mg to about 30 mg, about 20 mg to about 40 mg, about 20 mg to about 50 mg, about 25 mg to about 30 mg, about 25 mg to about 40 mg, about 25 mg to about 50 mg, about 30 mg to about 40 mg, about 30 mg to about 50 mg, or about 40 mg to about 50 mg. In some embodiments, the dose of the maqui berry extract comprises about 15 mg.

### Formulations and Compositions

Described herein are formulations and compositions comprising omega-3 fatty acids or esters thereof and maqui berry extract. In some embodiments, the omega-3 fatty acids or esters thereof comprise eicosapentaenoic acid (EPA), docosahexaenoic acid (DHA), or a combination thereof. In some embodiments, the omega-3 fatty acids or esters thereof comprise EPA. In some embodiments, the omega-3 fatty acids or esters thereof comprise DHA. In some embodiments, the maqui berry extract comprises maqui berry of the plant genus Aristotelia. In some embodiments, the maqui berry extract comprises maqui berry of the plant Aristotelia chilensis. In some embodiments, the formulations and compositions comprising omega-3 fatty acids or esters thereof and maqui berry extract comprise anthocyanins of the maqui berry.

In some embodiments, a ratio of omega-3 fatty acids or esters thereof to maqui berry extract varies from a range of about 5:1 to about 300:1. In some embodiments, the ratio of the omega-3 fatty acids or esters thereof to the maqui berry extract is at least or about 5:1, 10:1, 20:1, 25:1, 30:1, 35:1, 40:1, 45:1, 50:1, 60:1, 70:1, 80:1, 90:1, 100:1, 110:1, 120:1, 130:1, 140:1, 150:1, 160:1, 170:1, 180:1, 190:1, 200:1, 210:1, 220:1, 230:1, 240:1, 250:1, 260:1, 270:1, 280:1, 290:1, or 300:1. In some embodiments, the ratio of the omega-3 fatty acids or esters thereof to the maqui berry extract is about 12:1 to about 150:1. In some embodiments, the ratio of the omega-3 fatty acids or esters thereof to maqui berry extract are in a range of about 0.5:1 to about 300:1, 0.75:1 to about 275:1, about 1:1, to about 250, about 2:1 to about 225:1, about 3:1 to about 200:1, about 4:1 to about 175:1, about 5:1 to about 150:1, about 10:1 to about 125:1, about 20:1 to about 100:1, or about 40:1 to about 80:1. In some embodiments, the ratio of the omega-3 fatty acids or esters thereof to maqui berry extract is in a range of about 12:1 to about 150:1. In some embodiments, the ratio of the omega-3 fatty acids or esters thereof to the maqui berry extract is about 30:1 to about 150:1. In some embodiments, the ratio of the omega-3 fatty acids or esters thereof to the maqui berry extract is about 40:1 to about 120:1. In some embodiments, the ratio of the omega-3 fatty acids or esters thereof to the maqui berry extract is about 40:1 to about 80:1. In some embodiments, the ratio of the omega-3 fatty acids or esters thereof to the maqui berry extract is about 37.5:1.

Formulations and compositions comprising the omega-3 fatty acids or esters thereof and maqui berry extract, in some embodiments, comprise varying amounts of omega-3 fatty acids or esters thereof. The amount of omega-3 fatty acids or esters thereof, in some embodiments, is in a range of about 250 mg to about 1000 mg. In some embodiments, the amount of omega-3 fatty acids or esters thereof comprises at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of omega-3 fatty acids or esters thereof. In some embodiments, the amount of omega-3 fatty acids or esters thereof comprises about 100 mg to about 200 mg, about 100 mg to about 300 mg, about 100 mg to about 400 mg, about 100 mg to about 500 mg, about 100 mg to about 600 mg, about 100 mg to about 700 mg, about 100 mg to about 800 mg, about 100 mg to about 900 mg, about 100 mg to about 1,000 mg, about 100 mg to about 1,200 mg, about 200 mg to about 300 mg, about 200 mg to about 400 mg, about 200 mg to about 500 mg, about 200 mg to about 600 mg, about 200 mg to about 700 mg, about 200 mg to about 800 mg, about 200 mg to about 900 mg, about 200 mg to about 1,000 mg, about 200 mg to about 1,200 mg, about 300 mg to about 400 mg, about 300 mg to about 500 mg, about 300 mg to about 600 mg, about 300 mg to about 700 mg, about 300 mg to about 800 mg, about 300 mg to about 900 mg, about 300 mg to about 1,000 mg, about 300 mg to about 1,200 mg, about 400 mg to about 500 mg, about 400 mg to about 600 mg, about 400 mg to about 700 mg, about 400 mg to about 800 mg, about 400 mg to about 900 mg, about 400 mg to about 1,000 mg, about 400 mg to about 1,200 mg, about 500 mg to about 600 mg, about 500 mg to about 700 mg, about 500 mg to about 800 mg, about 500 mg to about 900 mg, about 500 mg to about 1,000 mg, about 500 mg to about 1,200 mg, about 600 mg to about 700 mg, about 600 mg to about 800 mg, about 600 mg to about 900 mg, about 600 mg to about 1,000 mg, about 600 mg to about 1,200 mg, about 700 mg to about 800 mg, about 700 mg to about 900 mg, about 700 mg to about 1,000 mg, about 700 mg to about 1,200 mg, about 800 mg to about 900 mg, about 800 mg to about 1,000 mg, about 800 mg to about 1,200 mg, about 900 mg to about 1,000 mg, about 900 mg to about 1,200 mg, or about 1,000 mg to about 1,200 mg. In some embodiments, the amount of omega-3 fatty acids or esters thereof comprises about 560 mg. In some embodiments, the amount of omega-3 fatty acids or esters thereof comprises at least or about 400 mg/g, 450 mg/g, 500 mg/g, 550 mg/g, 600 mg/g, 650 mg/g, 700 mg/g, 750 mg/g, 800 mg/g, 850 mg/g, 900 mg/g, 1000 mg/g or more than 1000 mg/g.

In some embodiments, formulations or compositions comprising omega-3 fatty acids or esters thereof and maqui berry extract comprise at least or about 20%, 25%, 30%, 35%, 40%, 45%, 50%, 55%, 60%, 65%, 70%, 75%, 80%, 85%, 90%, or more than 90% triglycerides. In some embodiments, formulations or compositions comprising omega-3 fatty acids or esters thereof and maqui berry extract comprise about 5% to about 100%, about 10% to about 90%, 20% to about 80%, or about 30% to about 70% of triglycerides. In some embodiments, formulations or compositions comprising omega-3 fatty acids or esters thereof and maqui berry extract comprise at most or about 1%, 1.5%, 2%, 2.5%, 3%, 3.5%, 4%, 4.5%, 5%, 6%, 7%, 8%, 9%, 10%, 12%, 14%, 16%, 18%, or 20% of oligomers. In some embodiments, formulations or compositions comprising omega-3 fatty acids or esters thereof and maqui berry extract comprise at most or about 20%, 25%, 30%, 35%, 40%, 45%, 50%, 55%, 60%, 65%, 70%, 75%, 80%, or 85% of partial glycerides. In some embodiments, formulations or compositions comprising omega-3 fatty acids or esters thereof and maqui berry extract comprise at most or about 50% of partial glycerides.

In some embodiments, the formulations and compositions comprising omega-3 fatty acids or esters thereof and maqui berry extract comprise eicosapentaenoic acid (EPA), docosahexaenoic acid (DHA), or a combination thereof. In some embodiments, omega-3 fatty acids or esters thereof comprise eicosapentaenoic acid (EPA), docosahexaenoic acid (DHA), or a combination thereof. In some embodiments, the omega-3 fatty acids or esters thereof comprise EPA. In some embodiments, the omega-3 fatty acids or esters thereof comprise DHA. In some embodiments, a ratio of the EPA to the DHA is in a range of about 0.7:1 to 5:1. In some embodiments, the ratio of the EPA to the DHA is at least or about 0.2:1, 0.25:1, 0.5:1, 0.7:1 0.75:1, 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1, 5:1, 5.25:1, 5.5:1, 5.75:1, or 6:1. In some embodiments, the ratio of the EPA to the DHA is about 0.2:1 to about 6:1, about 0.25:1 to about 5.75:1, about 5:1 to about 5.5:1, about 0.7:1 to about 5:1, about 0.75:1 to about 4.75:1, about 1:1 to about 4:1, or about 2:1 to about 3:1. In some embodiments, the ratio of the EPA to the DHA is about 2:1 to about 3:1. In some embodiments, the ratio of the EPA to the DHA is 2:1. In some embodiments, the ratio of the EPA to the DHA is 1.5:1. In some embodiments, the omega-3 fatty acids or esters thereof comprise at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of EPA. In some embodiments, the omega-3 fatty acids or esters thereof comprise about 10 mg to about 3000 mg, about 20 mg to about 2800 mg, about 40 mg to about 2400 mg, about 60 mg to about 2000 mg, about 80 mg to about 1600 mg, about 100 mg to about 1000, about 120 mg to about 800 mg, or about 200 mg to about 600 mg of EPA. In some embodiments, the omega-3 fatty acids or esters thereof comprise at least or about 300 mg/g, 350 mg/g, 400 mg/g, 450 mg/g, 500 mg/g, 550 mg/g, 600 mg/g, 650 mg/g, 700 mg/g or more of EPA. In some embodiments, the omega-3 fatty acids or esters thereof comprise at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of DHA. In some embodiments, the omega-3 fatty acids or esters thereof comprise about 10 mg to about 3000 mg, about 20 mg to about 2800 mg, about 40 mg to about 2400 mg, about 60 mg to about 2000 mg, about 80 mg to about 1600 mg, about 100 mg to about 1000, about 120 mg to about 800 mg, or about 200 mg to about 600 mg of DHA. In some embodiments, the omega-3 fatty acids or esters thereof comprise at least or about 100 mg/g, 150 mg/g, 200 mg/g, 250 mg/g, 300 mg/g, 350 mg/g, 400 mg/g, 450 mg/g or more of DHA. In some embodiments, the EPA or DHA is derived from sources including, but not limited to, fish, algae, squid, yeast, and vegetable sources.

In some embodiments, the formulations and compositions comprising omega-3 fatty acids or esters thereof and maqui berry extract comprise additional omega-3 fatty acids or esters thereof in addition to EPA and DHA. For example, the formulations and compositions comprise alpha-linolenic acid. In some embodiments, the additional omega-3 fatty acids or esters thereof comprise at least or about 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, or more than 100 mg.

In some embodiments, the formulations and compositions comprising omega-3 fatty acids or esters thereof and maqui berry extract comprise additional ingredients. In some embodiments, the formulations and compositions comprise beeswax, natural flavors, lecithin, rosemary extract, ascorbyl palmitate, or combinations thereof.

Provided herein, in some embodiments, are formulations and compositions comprising omega-3 fatty acids or esters thereof and maqui berry extract, wherein an amount of maqui berry extract is in a range of about 10 mg to about 300 mg. In some embodiments, the amount of the maqui berry extract comprises at least or about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg or more than 300 mg. In some embodiments, the amount of the maqui berry extract comprises about 5 mg to about 10 mg, about 5 mg to about 15 mg, about 5 mg to about 20 mg, about 5 mg to about 25 mg, about 5 mg to about 30 mg, about 5 mg to about 40 mg, about 5 mg to about 50 mg, about 10 mg to about 15 mg, about 10 mg to about 20 mg, about 10 mg to about 25 mg, about 10 mg to about 30 mg, about 10 mg to about 40 mg, about 10 mg to about 50 mg, about 15 mg to about 20 mg, about 15 mg to about 25 mg, about 15 mg to about 30 mg, about 15 mg to about 40 mg, about 15 mg to about 50 mg, about 20 mg to about 25 mg, about 20 mg to about 30 mg, about 20 mg to about 40 mg, about 20 mg to about 50 mg, about 25 mg to about 30 mg, about 25 mg to about 40 mg, about 25 mg to about 50 mg, about 30 mg to about 40 mg, about 30 mg to about 50 mg, or about 40 mg to about 50 mg. In some embodiments, the amount of the maqui berry extract comprises about 15 mg. In some embodiments, the amount of the maqui berry extract is in a range of about 20 mg to about 80 mg. In some embodiments, the amount of the maqui berry extract comprises about 60 mg.

In some embodiments, the composition and formulations described herein are administered to a subject by multiple administration routes, including but not limited to, parenteral (e.g., intravenous, subcutaneous, intramuscular), intracerebral, oral, intranasal, buccal, rectal, or transdermal administration routes. In some embodiments, the composition and formulations described herein are administered to a subject by oral administration.

In some embodiments, the formulations or compositions include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, cachets, tablet pills, gels, creams, emulsions, delayed release formulations, extended release formulations, pulsatile release formulations, multiparticulate formulations (e.g., nanoparticle formulations), and mixed immediate and controlled release formulations. Exemplary tablets include, but are not limited to, a suspension tablet, a fast-melt tablet, a bite-disintegration tablet, a rapid-disintegration tablet, an effervescent tablet, or a caplet. Capsules, in some embodiments, are made from animal-derived gelatin or plant-derived HPMC, or “sprinkle capsules”). In some embodiments, the capsule is a hard capsule. In some embodiments, the capsule is a soft capsule. In some embodiments, the capsule is a soft gel capsule.

In some embodiments, solid formulations comprising omega-3 fatty acid and maqui berry extract is rectangular, tubular, oblong, circular, round, oval in shape. In some embodiments, the solid formulations comprising omega-3 fatty acid and maqui berry extract is oblong in shape. In some embodiments, the solid formulations comprising omega-3 fatty acid and maqui berry extract is oval in shape.

In some embodiments, compositions or formulations comprising omega-3 fatty acids or esters thereof and maqui berry extract are administered as one or more soft gel capsules. In some embodiments, the soft gel capsules comprise at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of EPA. In some embodiments, the soft gel capsules comprise at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of DHA. In some embodiments, the soft gel capsules comprise about 560 mg of EPA and DHA. In some embodiments, the soft gel capsules comprise additional omega-3 fatty acids or esters thereof. In some embodiments, the soft gel capsules comprise about 10 mg to about 300 mg of maqui berry extract. In some embodiments, the soft gel capsules comprise about at least or about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg or more than 300 mg of maqui berry extract. In some embodiments, the soft gel capsules comprise about 20 mg to about 80 mg of maqui berry extract. In some embodiments, the soft gel capsules comprise about 60 mg of maqui berry extract.

In some embodiments, compositions or formulations comprising omega-3 fatty acids or esters thereof and maqui berry extract are administered in one or more soft gel capsules, wherein the one or more soft gel capsules comprise a range of about 50% to about 95% omega-3 fatty acids or esters thereof and about 1% to about 50% maqui berry extract. In some embodiments, the one or more soft gel capsules comprise at least or about 10%, 20%, 30%, 40%, 50%, 60%, 70%, 75%, 80%, 85%, 90%, 95% or more than 95% omega-3 fatty acids or esters thereof. In some embodiments, the one or more soft gel capsules comprise at least or about 10%, 20%, 30%, 40%, 50%, 60%, or more than 60% maqui berry extract.

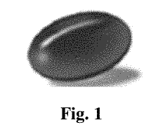

In some embodiments, compositions or formulations comprising omega-3 fatty acids or esters thereof and maqui berry extract are administered as one or more soft gel capsules, wherein the one or more soft gel capsules is rectangular, tubular, oblong, circular, round, oval in shape. In some embodiments, the one or more soft gel capsules is oblong. See e.g. FIG. 1. In some embodiments, the one or more soft gel capsules is oval. In some embodiments, a diameter of the one or more soft gel capsules comprises at least or about 0.1 cm, 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 6.6 cm, 7 cm, 7.5 cm, 8 cm, 8.5 cm, 9 cm, 10 cm, 11 cm, 12 cm, 13 cm, 14 cm, 15 cm, 16 cm, 17 cm, 18 cm, 19 cm, 20 cm, or more than 20 cm. In some embodiments, a width or height of the one or more soft gel capsules is at least or about 0.1 cm, 0.5 cm, 1 cm, 1.5 cm, 2 cm, 2.5 cm, 3 cm, 3.5 cm, 4 cm, 4.5 cm, 5 cm, 5.5 cm, 6 cm, 6.5 cm, 6.6 cm, 7 cm, 7.5 cm, 8 cm, 8.5 cm, 9 cm, 10 cm, 11 cm, 12 cm, 13 cm, 14 cm, 15 cm, 16 cm, 17 cm, 18 cm, 19 cm, 20 cm, or more than 20 cm. In some embodiments, the shape of the capsule is 5 oblong, 6 oblong, 7 oblong, 8 oblong, 9 oblong, 11 oblong, 14 oblong, 16 oblong, 17 oblong, 20 oblong, 22 oblong, or 24 oblong. In some embodiments, the shape of the capsule is 17.5 oblong. In some embodiments, the shape of the capsule is 20 oblong. In some embodiments, the shape of the capsule is 5 oval, 6 oval, 7 oval, 10 oval, 12 oval, 14 oval, 16 oval, 18 oval, 20 oval, 22 oval, or 24 oval. In some embodiments, the shape of the capsule is 14 oval. In some embodiments, the shape of the capsule is 5 round, 6 round, 7 round, 9 round, 10 round, 11 round, 12 round, 13 round, 14 round, 15 round, 16 round, 17 round, 18 round, 19 round, 20 round, 22 round, 24 round, or 28 round.

In some embodiments, the one or more soft gel capsules is purple. In some embodiments, the one or more soft gel capsules is red.

In some embodiments, compositions or formulations comprising omega-3 fatty acids or esters thereof and maqui berry extract are administered as one or more soft gel capsules, wherein the one or more soft gel capsules comprise a rupture of at most 15 minutes. In some embodiments, the rupture time is at most or about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, or 20 minutes. In some embodiments, the rupture time is in a range of about 1 minute to about 20 minutes, about 2 minutes to about 18 minutes, about 3 minutes to about 15 about, about 4 minutes to about 12 minutes, or about 6 minutes to about 10 minutes. In some embodiments, the rupture time at most or about 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, or more than 4 hours.

In some embodiments, compositions or formulations comprising omega-3 fatty acids or esters thereof and maqui berry extract are administered in one or more soft gel capsules, wherein the one or more soft gel capsules comprise a disintegration time of at most 45 minutes. In some embodiments, the disintegration time is at most or about 10 minute, 15 minutes, 20 minutes, 25 minutes, 30 minutes, 35 minutes, 40 minutes, 45 minutes, 50 minutes, 55 minutes, 1 hour, 2 hours, 3 hours, 4 hours or more than 4 hours. In some embodiments, the disintegration time is in a range of about 5 minutes to about 4 hours minutes, about 10 minutes to about 3 hours minutes, about 20 minutes to about 2 hours, about 25 minutes to about 1 hour, or about 30 minutes to about 50 minutes.

In some embodiments, the formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

In some embodiments, the formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some embodiments, the formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability.

In some embodiments, the formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some embodiments, the formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some embodiments, the formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™ sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Methods for Treatment

Described herein, in some embodiments, are methods, compositions, and formulations comprising omega-3 fatty acids or esters thereof and maqui berry extract for use in a treatment of a disease or disorder. In some embodiments, the disease or disorder is an ocular disease or disorder. In some embodiments, the disease or disorder is characterized by inflammation. For example, the disease or disorder is a degenerative disease, cancer, cardiovascular disease, diabetes, or arthritis.

Exemplary ocular diseases or disorder include, but are not limited to, inflammatory conjunctivitis, including allergic and giant papillary conjunctivitis, macular edema, uveitis, endophthalmitis, scleritis, corneal ulcers, dry eye syndrome, glaucoma, ischemic retinal disease, corneal transplant rejection, complications related to intraocular surgery such intraocular lens implantation and inflammation associated with cataract surgery, Behcet's disease, Stargardt disease, immune complex vasculitis, Fuch's disease, Vogt-Koyanagi-Harada disease, subretinal fibrosis, keratitis, vitreo-retinal inflammation, ocular parasitic infestation/migration, retinitis pigmentosa, cytomeglavirus retinitis and choroidal inflammation. In some embodiments, the ocular diseases or disorders that may be amendable to treatment by the methods and compositions provided herein may include, without limitation, ectropion, lagophthalmos, blepharochalasis, ptosis, xanthelasma of the eyelid, parasitic infestation of the eyelid, dermatitis of the eyelid, dacryoadenitis, epiphora, dysthyroid exophthalmos, conjunctivitis, scleritis, keratitis, corneal ulcer, corneal abrasion, snow blindness, arc eye, Thygeson's superficial punctate keratopathy, corneal neovascularization, Fuchs' dystrophy, keratoconus, keratoconjunctivitis sicca, iritis, uveitis, sympathetic ophthalmia, cataracts, chorioretinal inflammation, focal chorioretinal inflammation, focal chorioretinitis, focal choroiditis, focal retinitis, focal retinochoroiditis, disseminated chorioretinal inflammation, disseminated chorioretinitis, disseminated choroiditis, disseminated retinitis, disseminated retinochoroiditis, exudative retinopathy, posterior cyclitis, pars planitis, Harada's disease, chorioretinal scars, macula scars of posterior pole, solar retinopathy, choroidal degeneration, choroidal atrophy, choroidal sclerosis, angioid streaks, hereditary choroidal dystrophy, choroideremia, choroidal dystrophy (central arealor), gyrate atrophy (choroid), ornithinaemia, choroidal haemorrhage and rupture, choroidal haemorrhage (not otherwise specified), choroidal haemorrhage (expulsive), choroidal detachment, retinoschisis, retinal artery occlusion, retinal vein occlusion, hypertensive retinopathy, diabetic retinopathy, retinopathy, retinopathy of prematurity, macular degeneration, Bull's Eye maculopathy, epiretinal membrane, peripheral retinal degeneration, hereditary retinal dystrophy, retinitis pigmentosa, retinal haemorrhage, separation of retinal layers, central serous retinopathy, retinal detachment, macular edema, glaucoma—optic neuropathy, glaucoma suspect—ocular hypertension, primary open-angle glaucoma, primary angle-closure glaucoma, floaters, Leber's hereditary optic neuropathy, optic disc drusen, strabismus, ophthalmoparesis, progressive external ophthaloplegia, esotropia, exotropia, disorders of refraction and accommodation, hypermetropia, myopia, astigmastism, anisometropia, presbyopia, internal ophthalmoplegia, amblyopia, Leber's congenital amaurosis, scotoma, anopsia, color blindness, achromatopsia, maskun, nyctalopia, blindness, River blindness, micropthalmia, coloboma, red eye, Argyll Robertson pupil, keratomycosis, xerophthalmia, aniridia, sickle cell retinopathy, ocular neovascularization, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy-2 and lensectomy, vascular diseases, retinal ischemia, choroidal vascular insufficiency, choroidal thrombosis, neovascularization of the optic nerve, diabetic macular edema, cystoid macular edema, proliferative vitreoretinopathy, and neovascularization due to penetration of the eye or ocular injury. In some embodiments, the ocular disease or disorder is selected from a group consisting of allergies, glaucoma, cataract, corneal disease, vitreo-retinal diseases, optic nerve diseases or disorders, oculosystemic diseases and disorders, uvea diseases or disorders, or diabetic eye disease. In some embodiments, the ocular disease or disorder is Meibomian gland inflammation or Meibomian gland dysfunction. In some embodiments, the ocular disease or disorder is dry eye disease.

Provided herein are methods for administering to a subject with an ocular disease or disorder a daily dosage of omega-3 fatty acids or esters thereof and maqui berry extract. In some embodiments, a daily dosage of omega-3 fatty acids or esters thereof comprises about 500 mg to about 4000 mg. In some embodiments, a daily dosage of omega-3 fatty acids or esters thereof is at least or about 500 mg, 750 mg, 1000 mg, 1200 mg, 1400 mg, 1600 mg, 1800 mg, 2000 mg, 2200 mg, 2400 mg, 2600 mg, 2800 mg, 3000 mg, 3200 mg, 3400 mg, 3600 mg, 3800 mg, 4000 mg, or more than 4000 mg. In some embodiments, a daily dosage of omega-3 fatty acids or esters thereof is in a range of about 1000 mg to about 3000 mg. In some embodiments, a daily dosage of omega-3 fatty acids or esters thereof comprises about 2400 mg. In some embodiments, a daily dosage of omega-3 fatty acids or esters thereof comprises about 2250 mg. In some embodiments, a daily dosage of maqui berry extract comprises about 10 mg to about 300 mg. In some embodiments, the daily dosage of the maqui berry extract is at least or about 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg or more than 300 mg. In some embodiments, the daily dosage of the maqui berry extract is in a range of about 20 mg to about 80 mg. In some embodiments, the daily dosage of the maqui berry extract comprises about 60 mg.

Described herein, in some embodiments, are methods for administering to a subject with an ocular disease or disorder, wherein the daily dosage comprising omega-3 fatty acids or esters thereof and maqui berry extract is administered as one or more doses. In some embodiments, the dose of omega-3 fatty acids or esters thereof comprises at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of EPA. In some embodiments, the dose of omega-3 fatty acids or esters thereof is in a range of about 10 mg to about 3000 mg, about 20 mg to about 2800 mg, about 40 mg to about 2400 mg, about 60 mg to about 2000 mg, about 80 mg to about 1600 mg, about 100 mg to about 1000, about 120 mg to about 800 mg, or about 200 mg to about 600 mg of EPA. In some embodiments, the dose of omega-3 fatty acids or esters thereof comprises at least or about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or more than 1000 mg of DHA. In some embodiments, the dose of omega-3 fatty acids or esters thereof is in a range of about 10 mg to about 3000 mg, about 20 mg to about 2800 mg, about 40 mg to about 2400 mg, about 60 mg to about 2000 mg, about 80 mg to about 1600 mg, about 100 mg to about 1000, about 120 mg to about 800 mg, or about 200 mg to about 600 mg of DHA. In some embodiments, the dose of omega-3 fatty acids or esters thereof comprises a ratio of the EPA to the DHA that is in a range of about 0.7:1 to 5:1. In some embodiments, the ratio of the EPA to the DHA is at least or about 0.2:1, 0.25:1, 0.5:1, 0.7:1 0.75:1, 1:1, 1.25:1, 1.5:1, 1.75:1, 2:1, 2.25:1, 2.5:1, 2.75:1, 3:1, 3.25:1, 3.5:1, 3.75:1, 4:1, 4.25:1, 4.5:1, 4.75:1, 5:1, 5.25:1, 5.5:1, 5.75:1, or 6:1. In some embodiments, the ratio of the EPA to the DHA is about 2:1 to about 3:1. In some embodiments, the ratio of the EPA to the DHA is 2:1. In some embodiments, the ratio of the EPA to the DHA is 1.5:1. In some embodiments, a dose of the maqui berry extract is in a range of about 5 mg to about 100 mg. In some embodiments, the dose of the maqui berry extract comprises at least or about 5 mg, 10 mg, 15 mg, 20 mg, 25 mg, 30 mg, 35 mg, 40 mg, 45 mg, 50 mg, 55 mg, 60 mg, 65 mg, 70 mg, 80 mg, 85 mg, 90 mg, 95 mg, 100 mg, or more than 100 mg of the maqui berry extract. In some embodiments, the dose of the maqui berry extract comprises about 15 mg. In some embodiments, a ratio of omega-3 fatty acids or esters thereof to maqui berry extract varies from a range of about 5:1 to about 300:1. In some embodiments, the ratio the omega-3 fatty acids or esters thereof to the maqui berry extract is at least or about 5:1, 10:1, 20:1, 25:1, 30:1, 35:1, 40:1, 45:1, 50:1, 60:1, 70:1, 80:1, 90:1, 100:1, 110:1, 120:1, 130:1, 140:1, 150:1, 160:1, 170:1, 180:1, 190:1, 200:1, 210:1, 220:1, 230:1, 240:1, 250:1, 260:1, 270:1, 280:1, 290:1, or 300:1. In some embodiments, the ratio the omega-3 fatty acids or esters thereof to the maqui berry extract is about 12:1 to about 150:1. In some embodiments, the ratio the omega-3 fatty acids or esters thereof to the maqui berry extract is about 30:1 to about 120:1. In some embodiments, the ratio the omega-3 fatty acids or esters thereof to the maqui berry extract is about 40:1 to about 80:1. In some embodiments, the ratio the omega-3 fatty acids or esters thereof to the maqui berry extract is about 37.5:1.

In some embodiments, a plurality of doses comprising omega-3 fatty acids or esters thereof and maqui berry extract is administered to the subject. In some embodiments, 1 2, 3, 4, 5, 6, 7, 8, 9, or 10 different doses of the omega-3 fatty acids or esters thereof and maqui berry extract is administered.

In some embodiments, one or more doses comprising omega-3 fatty acids or esters thereof and maqui berry extract is administered to the subject over a period of time. In some embodiments, the period of time is 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, or more than 12 weeks. The period of time, in some embodiments, is about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, or more than 8 years. In some embodiments, the plurality of doses comprising omega-3 fatty acids or esters thereof and maqui berry extract is administered chronically. In some embodiments, one or more doses comprising omega-3 fatty acids or esters thereof and maqui berry extract is administered to the subject daily for a period of time.

Methods as described herein, in some embodiments comprise providing a dose of omega-3 fatty acids or esters thereof or maqui berry extract at least 1, 2, 3, 4, 5, 6, 7, 8, 9, 10, 11, 12 more than 12 times a day. In some embodiments, a dose of omega-3 fatty acids or esters thereof or maqui berry extract is administered at least 4 times a day. In some embodiments, a dose of omega-3 fatty acids or esters thereof or maqui berry extract is administered about once times a day. In some embodiments, a dose of omega-3 fatty acids or esters thereof or maqui berry extract is administered about 2 times a day. In some embodiments, a dose of omega-3 fatty acids or esters thereof or maqui berry extract is administered about 3 times a day. In some embodiments, a dose of omega-3 fatty acids or esters thereof or maqui berry extract is administered about 4 times a day. Administration, in some embodiments, is about every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In some embodiments, a time between administration is at least or about 1 hour, 2 hours, 3 hours, 4 hours, 5 hours, 6 hours, 7 hours, 8 hours, 9 hours, 10 hours, 11 hours, 12 hours, or more than 12 hours. In some embodiments, a time between administration is in a range of 0 hours to 24 hours, 1 hour to 23 hours, 2 hours to 22 hours, 3 hours to 21 hours, 4 hours to 20 hours, 5 hours to 19 hours, 6 hours to 18 hours, 7 hours to 17 hours, 8 hours to 16 hours, 9 hours to 15 hours, and 10 hours to 12 hours.

In some embodiments, administration of formulations or compositions as described herein comprising omega-3 fatty acids or esters thereof and maqui berry extract to the subject results in improvements in symptoms associated with the ocular disease or disorder. In some embodiments, the administration of omega-3 fatty acids or esters thereof and maqui berry extract inhibit disease progression.

Administration of formulations or compositions described herein comprising omega-3 fatty acids or esters thereof and maqui berry extract, in some embodiments, results in an improvement in the ocular disease or disorder or symptoms of the ocular disease or disorder by at least 10% as compared to a control. In some embodiments, the improvement is by at least or about 10%, 15%, 20%, 25%, 30%, 35%, 40%, 45%, 50%, 60%, 70%, 80%, 90%, or more than 90% as compared to a control. In some embodiments, the improvement is an increase by at least or about 10%, 15%, 20%, 25%, 30%, 35%, 40%, 45%, 50%, 60%, 70%, 80%, 90%, or more than 90% as compared to a control. In some embodiments, the improvement is at least or about 0.5, 1.0, 1.5, 2.0, 2.5, 3.0, 3.5, 4.0, 4.5, 5.0, 6.0, 7.0, 8.0, or more than 8.0-fold as compared to a control. In some embodiments, the control is the subject at baseline, a subject administered placebo, a subject administered omega-3 fatty acids or esters thereof only, or a subject administered maqui berry extract only.

Improvements in the ocular disease or disorder, in some embodiments, are determined using various endpoints. For example, reduction in symptoms, osmolarity, reduction in inflammation, a Schirmer's test, a tear breakup time test, or a corneal staining are used following treatment of compositions or formulations comprising omega-3 fatty acids or esters thereof and maqui berry extract. In some embodiments, inflammation is determined by the expression of one or more cytokines, chemokines, proteins, or other inflammatory markers involved in the ocular disease or disorder. Exemplary inflammatory markers include, but are not limited to, iNOS, COX2, TFN alpha, IL-4, IL-6, IL-8, IL-18, MMP1, MMP2, MMP9, MMP13, TGF-beta, In some embodiments, MMP-9 is measured.

### Kits/Article of Manufacture

Disclosed herein, in certain embodiments, are kits and articles of manufacture for use with one or more methods, compositions, and formulations described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include one or more compositions or formulations comprising omega-3 fatty acids or esters thereof and maqui berry extract, and optionally one or more pharmaceutical excipients described herein to facilitate the delivery of omega-3 fatty acids or esters thereof and maqui berry extract. Such kits further optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the compositions or formulations are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. Formulation of Omega-3 and Maqui Berry

Formulations of omega-3 fatty acids or esters thereof and maqui berry extract were generated in soft gel capsules. Specification and formulation information of the omega-3 fatty acids or esters thereof and maqui berry extract are seen in Table 1.

TABLE 1
Omega-3 and Maui Berry Formulations

| | Formula 1 | | Formula 2 | Formula 3 |
|---|---|---|---|---|
| Omega-3 Fatty | 887 mg +/− 5% | | | |
| Acids and Maqui | | | | |
| Berry | | | | |
| MaquiBright ™ | 15 | mg | | 15 mg |
| Aristotelia | | | | |
| chilensis Berry | | | | |
| EPA (as TG) | 400 | mg | 400 mg | |
| DHA (as TG) | 162.5 | mg | 162.5 mg | |
| EPA + DHA | 562.5 | mg | 562.5 mg | Ratio of |
| (as TG) | | | | 2:1-3:1 |
| Additional | 37.5 | mg | | |
| Omega-3 (as TG) | | | | |
| Total Omega-3s | 600 | mg | | 562.5 mg |
| (as TG) | | | | |
| Description | Reddish soft gel | | Purple soft gelatin | Violet Soft gel |
| | capsules containing | | capsule, oblong shape | |
| | oil paste | | containing yellow oily | |
| | | | liquid and one white | |
| | | | tablet immersed in the | |
| | | | liquid | |
| Soft gel Size and [symbol] | 17.5 Oblong | | 20 Oblong | 14 Oval |
| Average Fill Weight | 1022 mg +/− 5% | | | |
| Rupture Time | Max. 15 minutes | | | NMT 15 minutes |
| (on delivery) | | | | |
| Disintegration | | | Max. 45 minutes | |
| Acid Value | Max. 3 mg KOH/g | | Max. 3 mg KOH/g | |
| Peroxide Value | Max. 5 meq/kg | | Max. 5 meq/kg | |
| Anisidine Value | Max. 20 | | Max. 20 | |
| Absorbance | | | 0.73 Maximum | |
| | | | at 233 nm | |
| Unsaponifiable Matter | | | 2.0% Maximum | |
| Anthocyanin | | | 8.75 mg/cap minimum | NLT 8.75 mg/sg |
| Delphinidins | | | 6.25 mg/cap minimum | NLT 6.25 mg/sg |
| Lead | Less than 0.1 mg/kg | | | |
| Cadmium | Less than 0.1 mg/kg | | | |
| Mercury | Less than 0.1 mg/kg | | | |
| Arsenic (inorganic) | Less than 0.1 mg/kg | | | |
| PCBs (209 Congeners) | Max. 0.09 mg/kg | | | |
| Sum of Dioxins & | Max. 1.75 pg/g | | | |
| Furans (17) - TEQ | | | | |
| Sum of Dioxins & | Max. 3 pg/g | | | |
| Furans and Dioxin | | | | |
| like PCBs -TEQ | | | | |
| Content of Total | | | NLT 58% Expressed | |
| Omega-3 Fatty Acids | | | as triglycerides | |
| Content of oligomers | | | NMT 3.0% Oligomers | |
| and partial glycerides | | | NMT 50% of partial | |
| | | | glycerides | |
| Total aerobic | Max. 3,000 CFU/g | | 1000 ufc/g maximum | NMT 3,000 cfu/g |
| microbial count | | | | |
| Total Mold & | Max. 300 CFU/g | | Max. 100 CFU/g | NMT 300 cfu/g |
| Yeast Count | | | | |
| Escherichia coli | Negative/10 g | | Absent/1 g | Negative/10 g |
| Salmonella | Negative/10 g | | Absent/1 g | Negative/10 g |
| Staphylococcus aureus | Negative/10 g | | | Negative/10 g |
| Escherichia aureus | | | Absent/1 g | |
| Pseudomonas aeruginosa | | | Absent/1 g | |
| Total Coliforms | | | | <10 cfu/g |
| Container Closure | | | | Suitable container |
| System | | | | closure system to |
| | | | | achieve target soft gel |
| | | | | size sterility, shelf- |
| | | | | life and |

[symbol]

indicates data missing or illegible when filed

### Example 2. Clinical Study on Omega-3 and Maqui Berry Supplements for the Management of Dry Eye Disease

The effects of supplements comprising omega-3 fatty acids or esters thereof and maqui berry extract on dry eye disease were determined.

Fourteen subjects were included in the study. Inclusion criteria included subjects 18 years or older, males or females, subjects diagnosed with mild to moderate dry eye disease, tear breakup time (TBUT)<10 mm, subjects diagnosed with moderately severe signs of corneal staining, subjects willing and able to return for all study visits and to follow instructions from the Principal Investigator (PI) and staff, ability to swallow soft gels, and subjects taking aspirin daily, with unchanged dosage for the duration of the study. Exclusion criteria included subjects who are currently or at any time during their participation in the study taking any oral omega-3 fatty acids supplements or maqui berry supplements, current use of Lifitegrast (or equivalent), and subjects who are allergic to ingredients in the soft gels and/or have a history of allergy or hypersensitivity to fish and/or shellfish.

Five subjects received omega-3 fatty acids or esters thereof and maqui berry extract, 3 subjects received maqui berry extract alone, 4 subjects received omega-3 fatty acids or esters thereof alone, and 2 subjects received placebo. Table 2 shows the treatment information.

TABLE 2

| | Key Dietary Ingredient | |
|---|---|---|
| Dietary Supplement | (gms/capsule) | Dose |
| Omega-3 Fatty Acid | EPA: 816 mg and DHA- 544 | 3 times daily or as |
| Dietary Supplement | mg/capsule | directed by PI |
| Maqui Berry Extract | 60 mg/capsule | Once daily or as |
| Dietary Supplement | | directed by PI |
| Placebo Capsule | | Once daily or as |
| | | directed by PI |

Several endpoints were measured including subject questionnaires, changes in tear osmolarity values using TearLab® Osmolarity System, matrix metalloproteinase-9 (MMP-9) level assessment using the InflammaDry test, TBUT, Schirmer's Test, corneal staining evaluated with 2% fluorescein sodium using Oxford staining scale 0 to 5 (0: no staining; 5: severe staining). Table 3 shows a summary of the data collected after two weeks of the study.

TABLE 3

| | Omega-3 Fatty Acids | Omega-3 Fatty | | |
|---|---|---|---|---|
| | and Maqui Berry | Acids | Maqui Berry | Placebo |
| OSDI Reduction | 39.7% | 24.1% | 32.8% | 13.8% |
| in Symptoms | | | | |
| Osmolarity | 306.4 to 302.0 | 301.8 to 300.0 | 304.6 to 313.7 | 285.7 to 293.0 |
| MMP-9 | 20% Reduction | No Effect | No Effect | No Effect |
| Schirmer's Test | 21.0 to 16.6 | 15.2 to 15.2 | 14.6 to 18.5 | 19.0 to 24.0 |
| TBUT | 3.3 to 4.1 | 4.2 to 4.6 | 2.5 to 4.8 | 4.2 to 5.0 |
| Staining | 20.8% Reduction | 13.9% Reduction | No Effect | No Effect |

As seen in Table 3, subjects who received omega-3 fatty acids or esters thereof and maqui berry extract exhibited a reduction in patient discomfort and symptoms (ODSI), improved tear osmolarity, decreased inflammation associated with dry eye disease as measured by MMP-9, and decreased surface damage associated with dry eye disease (corneal staining) as compared to subjects who received omega-3 fatty acids or esters thereof alone, maqui berry extract alone, or placebo.

Data for individual subjects for the different groups and the average results were also determined. Table 4 shows the Average Clinical Study Results of subjects who received omega-3 fatty acids or esters thereof and maqui berry extract (“O3M”), omega-3 fatty acids or esters thereof only (“O3”), maqui berry extract, and placebo. Individual subject data for subjects who received omega-3 fatty acids or esters thereof and maqui berry extract (“O3M”) is shown in Table 5. Table 6 shows individual subject data for subjects who received omega-3 fatty acids or esters thereof only (“O3”). Table 7 shows individual subject data for subjects who received maqui berry extract. Table 8 shows individual subject data for subjects who received placebo.

TABLE 4
Average Clinical Study Results
O3M Clinical Study Results (Average)

| | | Study Assessments | | | | |
|---|---|---|---|---|---|---|
| | | O3M | | O3 | | Maqui Berry |
| | | Baseline | Week 2 | Baseline | Week 2 | Baseline |
| OSDI | | 50.25 | 30.3 | 55.99 | 31.88 | 54.17 |
| Score/Assessment | | | | | | |
| DED Score/ | Score | 0.557 | 0.443 | 0.584 | 0.452 | 0.625 |
| Assessment | Raw | 15.6 | 12.4 | 16.3 | 12.7 | 17.5 |

| | OD | OS | OD | OS | OD | OS | OD | OS | OD | OS |
|---|---|---|---|---|---|---|---|---|---|---|
| Changes in tear | 304 | 308.8 | 305 | 299 | 300.7 | 303 | 301.3 | 298.7 | 310.3 | 299 |
| osmolarity value | | | | | | | | | | |
| using TearLab ® | | | | | | | | | | |
| Osmolarity System | | | | | | | | | | |
| (mOsms/L) | | | | | | | | | | |
| InflammaDry test | 100 | 100 | 80 | 80 | 66.67 | 66.67 | 66.67 | 66.67 | 66.7 | 66.7 |
| (%) | | | | | | | | | | |
| Schirmer's Test | 18 | 24 | 20 | 13.2 | 16.33 | 14 | 17 | 13.33 | 16.67 | 12.67 |
| (mm) | | | | | | | | | | |
| TBUT (sec) | 3.2 | 3.4 | 4.4 | 3.8 | 4.67 | 3.67 | 4.67 | 4.67 | 2.3 | 2.7 |
| Corneal Staining | 2.4 | 2.4 | 1.8 | 2 | 2.3 | 2 | 2 | 1.7 | 1.3 | 1.7 |

| | | Study Assessments | | |
|---|---|---|---|---|
| | | Maqui Berry | Placebo | |
| | | Week 2 | Placebo | Week 2 |
| OSDI | | 36.37 | 34.38 | 20.55 |
| Score/Assessment | | | | |
| DED Score/ | Score | 0.423 | 0.322 | 0.268 |
| Assessment | Raw | 12 | 9 | 7.5 |

| | OD | OS | OD | OS | OD | OS |
|---|---|---|---|---|---|---|
| Changes in tear | 315 | 312.3 | 285.5 | 286 | 293 | 293 |
| osmolarity value | | | | | | |
| using TearLab ® | | | | | | |
| Osmolarity System | | | | | | |
| (mOsms/L) | | | | | | |
| InflammaDry test | 66.7 | 66.7 | 100 | 100 | 100 | 100 |
| (%) | | | | | | |
| Schirmer's Test | 18.33 | 18.67 | 21 | 17 | 27 | 21 |
| (mm) | | | | | | |
| TBUT (sec) | 5 | 4.7 | 4.5 | 4 | 5.5 | 4.5 |
| Corneal Staining | 1.3 | 1.7 | 1.5 | 2 | 1.5 | 2 |

TABLE 5
Individual Patient Data for Omega-3 Fatty Acid and Maqui Berry Supplement
Individual Patient Data: O3 & Maqui Berry (O3M) Dietary Supplement

| | | Study Assessments | | | | | |
|---|---|---|---|---|---|---|---|
| | | Subject 1 | | Subject 2 | | Subject 3 | |
| | | Baseline | Week 2 | Baseline | Week 2 | Baseline | Week 2 |
| OSDI | | 75 | 29.55 | 15.91 | 10.42 | 62.5 | 38.64 |
| Score/Assessment | | | | | | | |
| DED Score/ | Score | 0.893 | 0.393 | 0.071 | 0.071 | 0.714 | 0.643 |
| Assessment | Raw | 25 | 11 | 2 | 2 | 20 | 18 |

| | OD | OS | OD | OS | OD | OS | OD | OS | OD | OS | OD | OS |
|---|---|---|---|---|---|---|---|---|---|---|---|---|
| Changes in tear | 314 | 326 | 303 | 311 | 301 | 295 | 298 | 286 | 303 | 303 | 333 | 292 |
| osmolarity value | | | | | | | | | | | | |
| using TearLab ® | | | | | | | | | | | | |
| Osmolarity System | | | | | | | | | | | | |
| (mOsms/L) | | | | | | | | | | | | |
| InflammaDry test | + | + | + | + | + | + | + | + | + | + | + | + |
| (%) | | | | | | | | | | | | |
| Schirmer's Test | 31 | >35 | 27 | 9 | 28 | 28 | 27 | 17 | 14 | 16 | 24 | 21 |
| (mm) | | | | | | | | | | | | |
| TBUT (sec) | <1 | <1 | 3 | 3 | <3 | <4 | 4 | 4 | <3 | <4 | 3 | 2 |
| Corneal Staining | 2 | 2 | 2 | 2 | 2 | 2 | 2 | 2 | 3 | 3 | 2 | 3 |

| | | Study Assessments | | | |
|---|---|---|---|---|---|
| | | Subject 5 | | Subject 6 | |
| | | Baseline | Week 2 | Baseline | Week 2 |
| OSDI | | 77 | 58.33 | 20.83 | 14.58 |
| Score/Assessment | | | | | |
| DED Score/ | Score | 0.679 | 0.679 | 0.429 | 0.429 |
| Assessment | Raw | 19 | 19 | 12 | 12 |

| | OD | OS | OD | OS | OD | OS | OD | OS |
|---|---|---|---|---|---|---|---|---|
| Changes in tear | 298 | 311 | 308 | 307 | Below | Below | 283 | Below |
| osmolarity value | | | | | range | range | | range |
| using TearLab ® | | | | | | | | |
| Osmolarity System | | | | | | | | |
| (mOsms/L) | | | | | | | | |
| InflammaDry test | + | + | − | − | + | + | + | + |
| (%) | | | | | | | | |
| Schirmer's Test | 8 | 15 | 5 | 5 | 9 | 26 | 17 | 14 |
| (mm) | | | | | | | | |
| TBUT (sec) | 5 | 4 | 6 | 5 | <4 | <4 | 6 | 5 |
| Corneal Staining | 2 | 2 | 1 | 1 | 3 | 3 | 2 | 2 |

TABLE 6
Individual Patient Data for Omega-3 Fatty Acid Supplement
Individual Patient Data: O3 Dietary Supplement

| | | Study Assessments | | | | | | | |
|---|---|---|---|---|---|---|---|---|---|
| | | Subject 4 | | Subject 10 | | Subject 11 | | Subject 12 | |
| | | Baseline | Week 2 | Baseline | Week 2 | Baseline | Week 2 | Baseline | Week 2 |
| OSDI | | 68.75 | 35.42 | 65.91 | 37.5 | 85.42 | Note: | 33.33 | 22.73 |
| Score/Assessment | | | | | | | Patient | | |
| DED Score/ | Score | 0.679 | 0.601 | 0.643 | 0.321 | 0.786 | developed | 0.429 | 0.429 |
| Assessment | Raw | 19 | 17 | 18 | 9 | 22 | influenza | 12 | 12 |

| | OD | OS | OD | OS | OD | OS | OD | OS | OD | OS | | OD | OS | OD | OS |
|---|---|---|---|---|---|---|---|---|---|---|---|---|---|---|---|
| Changes in tear | 293 | 303 | 299 | 298 | 298 | 291 | 290 | 289 | 290 | 285 | and had | 311 | 315 | 315 | 309 |
| osmolarity value | | | | | | | | | | | to be | | | | |
| using TearLab ® | | | | | | | | | | | dropped | | | | |
| Osmolarity System | | | | | | | | | | | from the | | | | |
| (mOsms/L) | | | | | | | | | | | study | | | | |
| InflammaDry test | + | + | + | + | + | + | + | + | + | + | | − | − | N/A | N/A |
| (%) | | | | | | | | | | | | | | | |
| Schirmer's Test | 17 | 16 | 17 | 14 | 5 | 9 | 12 | 10 | 17 | 13 | | 27 | 17 | 22 | 16 |
| (mm) | | | | | | | | | | | | | | | |
| TBUT (sec) | <4 | <5 | 3 | 3 | 5 | 3 | 6 | 7 | 4 | 4 | | 5 | 3 | 5 | 4 |
| Corneal Staining | 3 | 3 | 2 | 2 | 1 | 1 | 1 | 1 | 3 | 3 | | 3 | 2 | 3 | 2 |

TABLE 7
Individual Patient Data for Maqui Berry Supplement
Individual Patient Data: Maqui Berry Dietary Supplement

| | | Study Assessments | | | | | |
|---|---|---|---|---|---|---|---|
| | | Subject 7 | | Subject 8 | | Subject 9 | |
| | | Baseline | Week 2 | Baseline | Week 2 | Baseline | Week 2 |
| OSDI | | 50 | 45.83 | 54.17 | 29.17 | 58.33 | 34.1 |
| Score/Assessment | | | | | | | |
| DED Score/ | Score | 0.643 | 0.464 | 0.714 | 0 | 0.607 | 0.393 |
| Assessment | Raw | 18 | 13 | 20 | 0 | 17 | 11 |

| | OD | OS | OD | OS | OD | OS | OD | OS | OD | OS | OD | OS |
|---|---|---|---|---|---|---|---|---|---|---|---|---|
| Changes in tear | 319 | 313 | 322 | 316 | 316 | 290 | 319 | 314 | 296 | 294 | 304 | 307 |
| osmolarity value | | | | | | | | | | | | |
| using TearLab ® | | | | | | | | | | | | |
| Osmolarity System | | | | | | | | | | | | |
| (mOsms/L) | | | | | | | | | | | | |
| InflammaDry test | − | − | N/A | N/A | + | + | + | + | + | + | + | + |
| (%) | | | | | | | | | | | | |
| Schirmer's Test | 15 | 8 | 18 | 15 | 16 | 18 | 14 | 17 | 19 | 12 | 23 | 24 |
| (mm) | | | | | | | | | | | | |
| TBUT (sec) | <1 | <1 | 4 | 3 | <2 | <2 | 6 | 4 | <4 | <5 | 5 | 7 |
| Corneal Staining | 2 | 2 | 2 | 2 | 1 | 2 | 1 | 2 | 1 | 1 | 1 | 1 |

TABLE 8
Individual Patient Data for Placebo
Individual Patient Data: Placebo Dietary Supplement

| | | Study Assessments | | | |
|---|---|---|---|---|---|
| | | Subject 13 | | Subject 14 | |
| | | Baseline | Week 2 | Baseline | Week 2 |
| OSDI | | 29.17 | 22.92 | 39.58 | 18.18 |
| Score/Assessment | | | | | |
| DED Score/ | Score | 0.393 | 0.357 | 0.25 | 0.179 |
| Assessment | Raw | 11 | 10 | 7 | 5 |

| | OD | OS | OD | OS | OD | OS | OD | OS |
|---|---|---|---|---|---|---|---|---|
| Changes in tear | 293 | Below | 297 | 296 | 278 | 286 | 289 | 290 |
| osmolarity value | | range | | | | | | |
| using TearLab ® | | | | | | | | |
| Osmolarity System | | | | | | | | |
| (mOsms/L) | | | | | | | | |
| InflammaDry test | + | + | + | + | + | + | + | + |
| (%) | | | | | | | | |
| Schirmer's Test | 20 | 18 | 23 | 16 | 22 | 16 | 31 | 26 |
| (mm) | | | | | | | | |
| TBUT (sec) | 4 | 4 | 4 | 3 | 5 | 4 | 7 | 6 |
| Corneal Staining | 2 | 2 | 2 | 2 | 1 | 2 | 1 | 2 |

As seen in the above data, there was an improvement in tear volume among the subjects assigned to the maqui group and an increase in tear film hyperosmolarity. All but one subject in the maqui group tested positive for MMP-9 at baseline and remained positive through week 2. The O3M group experienced the greatest improvement in osmolarity scores. This may contribute to the improvement observed in corneal staining, and is further supported by the improvement in TBUT in the maqui group, while no improvement was observed in corneal staining, which would be indicative of ocular inflammation.

The data shows that omega-3 fatty acids or esters thereof have anti-inflammatory properties and maqui berry have the ability to upregulate lacrimal gland secretion, and that there is a combined benefit of omega-3 fatty acids or esters thereof and maqui berry administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
(a) omega-3 fatty acids or esters thereof; and
(b) maqui berry extract,
wherein a ratio of the omega-3 fatty acids or esters thereof to the maqui berry extract is about 12:1 to about 150:1.

2. The composition of claim 1, wherein the ratio of the omega-3 fatty acids or esters thereof to the maqui berry extract is about 30:1 to about 120:1.

3. The composition of claim 1, wherein the ratio of the omega-3 fatty acids or esters thereof to the maqui berry extract is about 40:1 to about 80:1.

4. The composition of claim 1, wherein the composition is formulated for oral administration.

5. The composition of claim 1, wherein the composition is formulated as a capsule, cachet, or tablet.

6. The composition of claim 5, wherein the capsule is a soft gel capsule.

7. The composition of claim 1, wherein the omega-3 fatty acids or esters thereof comprise eicosapentaenoic acid (EPA) or docosahexaenoic acid (DHA).

8. The composition of claim 1, wherein the omega-3 fatty acids or esters thereof comprise eicosapentaenoic acid (EPA) and docosahexaenoic acid (DHA).

9. The composition of claim 8, wherein a ratio of the EPA to the DHA is in a range of 0.7:1 to 5:1.

10. The composition of claim 8, wherein a ratio of the EPA to the DHA is in a range of 1:1 to 4:1.

11. The composition of claim 8, wherein a ratio of the EPA to the DHA is in a range of 2:1 to 3:1.

12. The composition of claim 8, wherein a ratio of the EPA to the DHA is 2:1.

13. The composition of claim 8, wherein a ratio of the EPA to the DHA is 1.5:1.

14. A method of treating an ocular disease or disorder in a subject comprising:
administering to the subject omega-3 fatty acids or esters thereof and maqui berry extract, wherein a ratio of the omega-3 fatty acids or esters thereof to the maqui berry extract is about 12:1 to about 150:1.

15. The method of claim 14, wherein the ocular disease or disorder is selected from a group consisting of allergies, glaucoma, cataract, corneal disease, vitreo-retinal diseases, optic nerve diseases or disorders, oculosystemic diseases and disorders, uvea diseases or disorders, and diabetic eye disease.

16. The method of claim 14, wherein the ocular disease or disorder is dry eye.

17. The method of claim 14, wherein a dose of the omega-3 fatty acids or esters thereof is administered to the subject one time to four times a day.

18. The method of claim 14, wherein a dose of the maqui berry extract is administered to the subject one time to four times a day.

19. The method of claim 14, wherein the ratio of the omega-3 fatty acids or esters thereof to the maqui berry extract is about 30:1 to about 120:1.

20. The method of claim 14, wherein the ratio of the omega-3 fatty acids or esters thereof to the maqui berry extract is about 40:1 to about 80:1.

21. The method of claim 14, wherein the omega-3 fatty acids or esters thereof comprise eicosapentaenoic acid (EPA) or docosahexaenoic acid (DHA).

22. The method of claim 14, wherein the omega-3 fatty acids or esters thereof comprise eicosapentaenoic acid (EPA) and docosahexaenoic acid (DHA).

23. The method of claim 22, wherein a ratio of the EPA to the DHA is in a range of 0.7:1 to 5:1.

24. The method of claim 22, wherein a ratio of the EPA to the DHA is in a range of 1:1 to 4:1.

25. The method of claim 22, wherein a ratio of the EPA to the DHA is in a range of 2:1 to 3:1.

26. The method of claim 22, wherein a ratio of the EPA to the DHA is 2:1.

27. The method of claim 22, wherein a ratio of the EPA to the DHA is 1.5:1.

28. The method of claim 14, wherein the omega-3 fatty acids or esters thereof and the maqui berry extract are formulated for oral administration.

29. The method of claim 14, wherein the omega-3 fatty acids or esters thereof and the maqui berry extract are formulated as a capsule, cachet, or tablet.

30. The method of claim 29, wherein the capsule is a soft gel capsule.
